(19) United States
(12) Patent Application Publication
(10) Pub. No.: US 2007/0270935 A1
(43) Pub. Date: Nov. 22, 2007

(54) DUAL BALLOON CATHETER AND DEPLOYMENT OF SAME

(75) Inventors: Richard R. Newhauser, San Francisco, CA (US); Howard H. Huang, Santa Clara, CA (US)

Correspondence Address: BEYER WEAVER LLP/Abbott Vascular Devices, P.O. BOX 70250, OAKLAND, CA, 94612-0250, US

(73) Assignee: ABBOTT LABORATORIES, Abbott Park, IL (US)

(21) Appl. No.: 11/747,158

(22) Filed: May 10, 2007

Related U.S. Application Data
(60) Provisional application No. 60/802,010, filed on May 18, 2006.

(51) Int. Cl. A61F 2/84

(52) U.S. Cl. 623/1.11; 606/194

(57) ABSTRACT

Disclosed are catheter apparatus and methods for deploying a stent in a bifurcated vessel while minimizing flow problems in the branch vessel. In general, the catheter includes at least one non-compliant inflation structure for pushing a stent against a vessel wall in a main vessel and a compliant inflation structure for forming an opening in the stent across an opening in a branch vessel. Ideally, flow reduction caused by the stent is minimized due to the stent opening at the branch vessel. Additionally, other medical devices, such as a guide wire or a second stent, may be easily inserted through the opening in the stent into the branch vessel.

### CROSS REFERENCE TO RELATED PATENT APPLICATION

This application claims priority of co-pending U.S. Provisional Patent Application No. 60/802,010, entitled DUAL BALLOON CATHETER AND DEPLOYMENT OF SAME, by Richard R Newhauser, filed 18 May 2006, under 35 U.S.C. §119(e), and which application is incorporated herein by reference in its entirety for all purposes.

### BACKGROUND OF THE INVENTION

The present invention relates generally to catheters. More particularly, the present invention relates to catheters that are positioned in the vicinity of vessel bifurcations.

A type of endoprosthesis device, commonly referred to as a stent, may be placed or implanted within a vein, artery or other tubular body organ for treating occlusions, stenoses, or aneurysms of a vessel by reinforcing the wall of the vessel or by expanding the vessel. Stents have been used to treat dissections in blood vessel walls caused by balloon angioplasty of the coronary arteries as well as peripheral arteries and to improve angioplasty results by preventing elastic recoil and remodeling of the vessel wall. Two randomized multicenter trials have recently shown a lower restenosis rate in stent treated coronary arteries compared with balloon angioplasty alone (Serruys, P W et al. New England Journal of Medicine 331: 489-495, 1994, Fischman, D L et al. New England Journal of Medicine 331:496-501, 1994). Stents have been successfully implanted in the urinary tract, the bile duct, the esophagus and the tracheo-bronchial tree to reinforce those body organs, as well as implanted into the neurovascular, peripheral vascular, coronary, cardiac, and renal systems, among others. The term “stent” as used in this Application is a device that is intraluminally implanted within bodily vessels to reinforce collapsing, dissected, partially occluded, weakened, diseased or abnormally dilated or small segments of a vessel wall.

In some applications the targeted region of a vessel may be at a location where the vessel bifurcates. FIG. 1 is a diagrammatic representation of a side view, in cross-section, of a bifurcated vessel 100 with a stent apparatus 106. As shown, the stent 106 is positioned in a main branch 102 of the bifurcated vessel 100, while a portion 108 of the stent is positioned over the opening to the side branch 104.

Insertion of a stent into a main vessel spanning the bifurcation point may pose a limitation to blood flow and access to the side branch vessel. The term “jail” or “jailing” is often used to describe the struts of the stent spanning across the opening of the branch vessel. In this regard, the tubular slotted hinged design of the Palmaz-Schatz intracoronary stent, in particular, is felt to be unfavorable for lesions with a large side branch and is generally believed to pose a higher risk of side branch vessel entrapment where the stent prevents or limits access to the side branch. Id.

In situations where the main or side branch has been stented and the stent is positioned over the bifurcation, it may be difficult to pass a wire through the stent structure for treating the other vessel, if desired. As shown in FIG. 1, a physician attempts to position wire 110 into side branch 104 by moving the curved tip portion of the wire along direction 112. However, the wire 110 is trapped by stent portion 108 and cannot advance into the side branch 104.

Plaque shifting during deployment of the stent may also occur. Plaque shifting may then occlude (or partially occlude) the other vessel. FIG. 2A is a diagrammatic side view, in cross section, of a bifurcated vessel 200 having a main branch 202 and a side branch 204 with plaque deposits 206 formed around the side branch opening. As shown, plaque 206a and 206b is deposited in two areas that are proximate to the side branch opening 207. FIG. 2B illustrates the bifurcated vessel 200 of FIG. 2A after stent deployment and resulting plaque displacement. Stent 208 is positioned in the main artery 202 and across the opening of the side branch 204. After or during deployment of the stent 208, one or more plaque deposits may shift into the stent. Plaque portions 206c and 206d are entrapped in the stent portion 207 that is positioned over the branch vessel opening. These shifted plaque portions 206c and 206d impede blood flow through such opening.

Accordingly, improved mechanisms for deploying a stent in the vicinity of a bifurcated vessel are needed.

### SUMMARY OF THE INVENTION

Accordingly, catheter apparatus and methods for deploying a stent in a bifurcated vessel while minimizing flow problems in the branch vessel are provided. In general, the catheter includes at least one non-compliant inflation structure for pushing a stent against a vessel wall in a main vessel and a compliant inflation structure for forming an opening in the stent across an opening in a branch vessel. Ideally, flow reduction caused by the stent is minimized due to the stent opening at the branch vessel. Additionally, other medical devices, such as a guide wire or a second stent, may be easily inserted through the opening in the stent into the branch vessel.

In one embodiment, a catheter is disclosed. The catheter includes an elongate flexible tubular member having a plurality of lumens including a guide lumen and at least one inflation lumen. The flexible tubular member is sized for insertion in a body vessel. The catheter further includes a first inflatable structure carried by a distal portion of the flexible tubular member. The first inflatable structure is in fluid communication with at least one of the inflation lumen, and the first inflatable structure is formed from a substantially non-compliant material. The catheter further includes a second inflatable structure carried by a distal portion of the flexible tubular member, and the second inflatable structure is in fluid communication with at least one of the inflation lumen. The second inflatable structure is formed from a substantially, compliant material.

In a specific implementation, the second inflatable structure covers a portion of the first inflatable structure. In another aspect, the second inflatable structure forms a toroid shape that encompasses a portion of the first inflatable structure. In a further aspect, the second inflatable structure forms a partial toroid shape that partially encompasses the first inflatable structure. In another embodiment, the first and second inflation structures are in fluid communication with the same inflation lumen. In a further aspect, the same inflation lumen is positioned inside the first inflation structure and the inside of the first inflation structure is in fluid communication with the inside of the second inflation structure. In yet a further aspect, the first inflation structure has one or more opening(s) that are in fluid communication with an interior of the second inflation structure. For instance, each opening is aligned with a corresponding opening in the second inflation structure and wherein a portion of the first inflation structure is adhered to a portion of the second inflation structure so as to form a seal around each of the openings of the first and second inflation structures. In another aspect, the openings of the first inflation structures are radially distributed around the hole of the second inflation structure.

In another implementation, the first inflation structure is in fluid communication with a first inflation lumen and the second inflation structure is in fluid communication with a second inflation lumen that differs from the first inflation lumen. In a further aspect, first inflatable structure has a cylindrical shape and the second inflatable structure is formed on an external, curved surface portion of the first inflatable structure. In this aspect, the first inflation lumen extends to inside an end of the first inflation structure and the second inflation lumen branches from the tubular member and extends to the second inflatable structure. In another embodiment, the catheter also includes a plurality of markers for detecting a position of the first inflatable structure and a position of the second inflatable structure. In a specific implementation, the guide lumen is sized for insertion of a guide wire or exchanger.

In another embodiment, the invention pertains to a method for inserting a stent into a body vessel using a catheter. The catheter comprises a first inflatable structure carried by a distal portion of a flexible tubular member and a second inflatable structure carried by a distal portion of the flexible tubular member. The first inflatable structure is formed from a substantially, non-compliant material and the second inflatable structure is formed from a substantially, compliant material. The catheter is positioned in a main vessel such that the first inflatable structure is positioned along a target area of the main vessel and the second inflatable structure is positioned across an orifice of a branch vessel that branches from the main vessel. The first inflatable structure is inflated to thereby expand the stent against the target area of the main vessel, and the second inflatable structure is inflated to thereby push a portion of the stent to form an opening in the stent portion at the orifice of the branch vessel. The first and second inflatable structures are then deflated and removing the catheter from the main vessel.

In a specific aspect, a second stent is deployed into the branch vessel through the opening of the first stent. In another aspect, the operation of inflating the first inflatable structure is performed substantially, simultaneously with the operation of inflating the second inflatable structure. Alternatively, the operation of inflating the first inflatable structure is performed prior to the operation of inflating the second inflatable structure.

These and other features and advantages of the present invention will be presented in more detail in the following specification of the invention and the accompanying figures which illustrate by way of example the principles of the invention.

### BRIEF DESCRIPTION OF THE DRAWINGS

FIG. 1 is a diagrammatic representation of a side view, in cross-section, of a bifurcated vessel with a stent apparatus.

FIG. 2A is a diagrammatic side view, in cross section, of a bifurcated vessel having a main branch and a side branch with plaque formed around the side branch opening.

FIG. 2B illustrates the bifurcated vessel of FIG. 2A after stent deployment and resulting plaque displacement.

FIG. 3A is a diagrammatic representation of a catheter having a dual balloon mechanism with single inflation lumen in accordance with one embodiment of the present invention.

FIG. 3B is a close up of the dual balloon mechanism of FIG. 3A.

FIG. 3C illustrates the plurality of openings in the non-compliant inflation structure for fluid communication between the non-compliant and compliant balloons in accordance with one implementation of the present invention.

FIGS. 3D through 3H illustrate different lumen configurations for the catheter of FIGS. 3A through 3C in accordance with various embodiments of the present invention.

FIG. 4A is a diagrammatic representation of a catheter having a dual balloon mechanism with dual inflation lumen in accordance with one embodiment of the present invention.

FIG. 4B is a close up of the dual balloon mechanism of FIG. 4A.

FIG. 4C is a cross section of the catheter of FIG. 4A along line A-A in accordance with one embodiment of the present invention.

FIGS. 4D through 4H illustrate different lumen configurations for the catheter of FIGS. 4A and 4B in accordance with various embodiments of the present invention.

FIG. 5 is a flowchart illustrating a procedure for deploying a stent using a dual balloon system in accordance with one embodiment of the present invention.

FIG. 6A is a diagrammatic representation illustrating placement of a catheter with a dual balloon and stent within a bifurcated vessel in accordance with one embodiment of the present invention.

FIG. 6B illustrates inflation and expansion of the non-compliant balloon and overlying stent of FIG. 6A.

FIG. 6C illustrates inflation and expansion of the compliant balloon.

FIG. 6D illustrates a main vessel into which a stent has been deployed in a bifurcated vessel so that the stent opens across the branch vessel in accordance with one embodiment of the present invention.

FIGS. 7A and 7B illustrate an alternative stent and balloon arrangement for deploying in a bifurcated vessel.

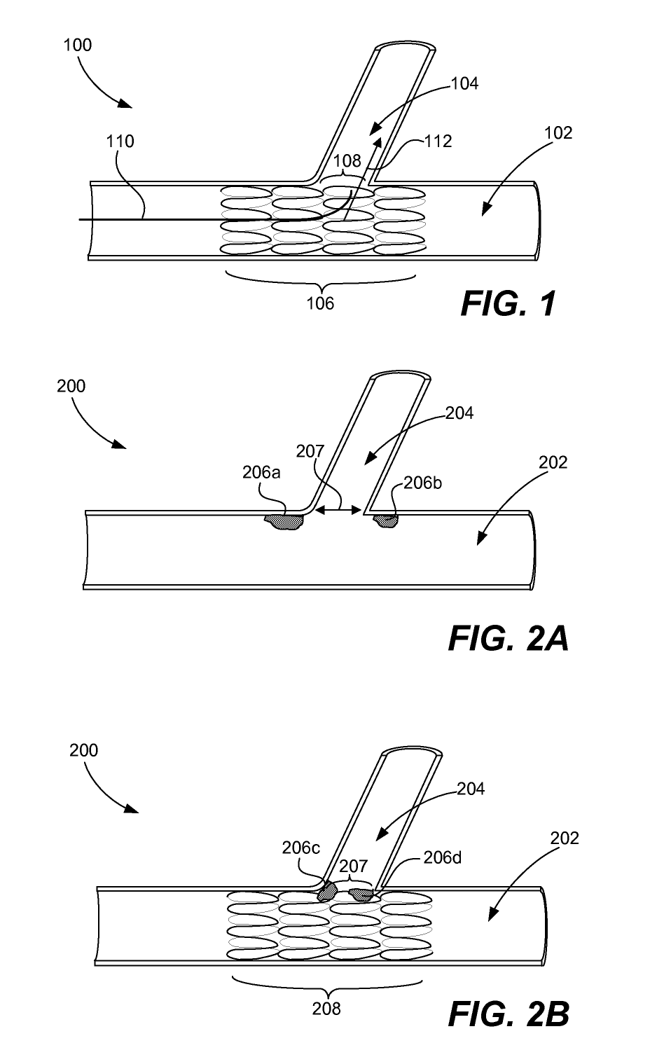

### DETAILED DESCRIPTION OF THE SPECIFIC EMBODIMENTS

Reference will now be made in detail to a specific embodiment of the invention. An example of this embodiment is illustrated in the accompanying drawings. While the invention will be described in conjunction with this specific embodiment, it will be understood that it is not intended to limit the invention to one embodiment. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

In general, a dual balloon catheter for use in a bifurcated body vessel is provided. The catheter includes a substantially, non-compliant balloon and a substantially, compliant balloon. A substantially, non-compliant or balloon is formed from a material such as Nylon (e.g., Nylon 12), Pebax, PEEK (Polyetheretherketone), PET (Polyethylene Terephthalate), or PTFE (Polytetrafluoroethylene). A semi-compliant or substantially compliant balloon is formed from a material such as Pebax (e.g., Pebax 7033 or a Pebax Blend), C-Flex, Latex, Polyethylene, PE600 (Polyethylene600), Urethane, or Silicone. A stent is crimped over both the non-compliant and compliant balloons, and the catheter is positioned in a main vessel with the compliant balloon being positioned at a branch vessel opening. The non-compliant balloon is designed to expand the stent against a target area of the main vessel, and the compliant balloon is structured to push against a portion of the stent to form an opening in the stent at the branch vessel. Non-compliant balloons tend to have a predetermined cylindrical shape (or other predetermined shape) that limits the expansion of the stent to a specified diameter. Expanding the stent in the main vessel with a non-compliant balloon ensures that the stent is expanded to vessel diameter and limits over expanding the stent. Whereas, the compliant balloon has less of a predetermined size or shape and can freely expand into the branch vessel clearing the struts from the orifice of the vessel to thereby force the struts against the walls of the branch vessel.

The dual balloon catheter of the present invention may have any suitable construction that achieves stent expansion against a target portion of a main vessel and also opening a portion of such stent at the orifice of a branch vessel. The opening in the stent at the branch vessel orifice prevents or minimizes the stent forming a barrier to the flow through such branch vessel. Additionally, a second stent may be more easily inserted into the branch vessel through the stent opening of the stent deployed in the main vessel.

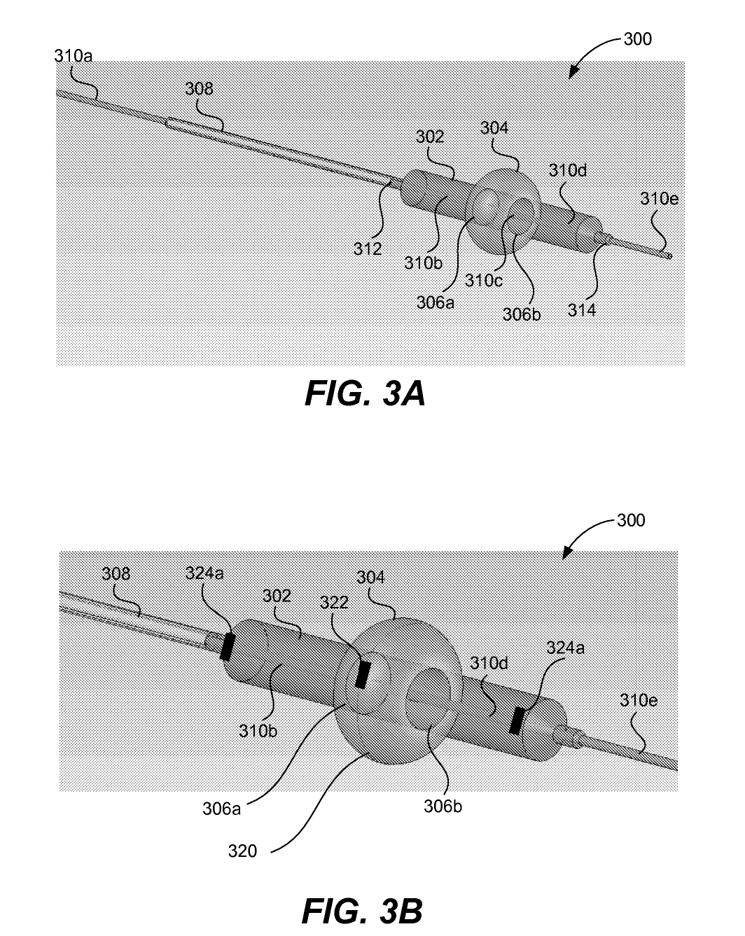

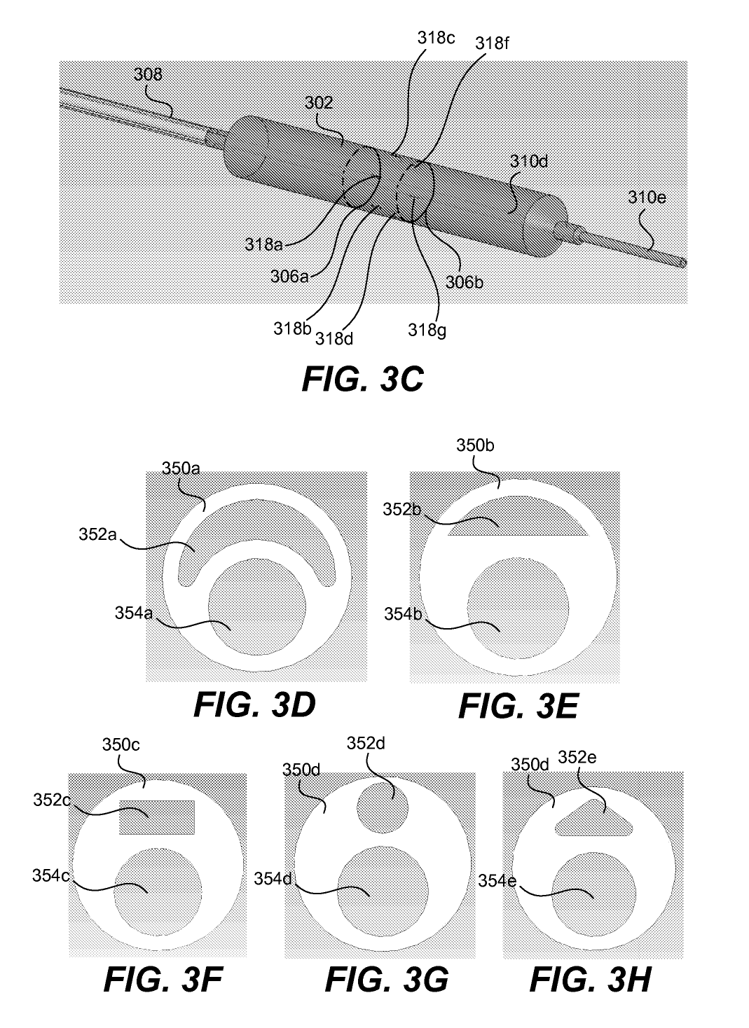

FIG. 3A is a diagrammatic representation of a catheter 300 having a dual balloon mechanism with single inflation lumen in accordance with one embodiment of the present invention. FIG. 3B is a close up of the dual balloon mechanism of FIG. 3A. The catheter 300 has an elongated, flexible tubular member 308 that is sized suitably for insertion in a vessel of interest. As will be appreciated by those familiar with the art, only the distal, working end of the catheter 300 is shown in these figures. The length and size of the catheter 300 will typically depend on its desired application and the proximal end of the catheter would typically be outfitted with a suitable handle and ports, valves and other structures for controlling the working (distal) end of the catheter.

In the described embodiment, the catheter is designed for deployment in vascular vessels including coronary vessels. However, in other embodiments, the catheter may be designed for insertion in any body vessel or tubular structure of the body. The flexible tubular member 308 includes at least one guide lumen and one inflation (e.g., fluid supply) lumen. As shown, the bottom portion of the flexible tubular member 308 has been removed to show the bottom portion of guide tube 310a. However, the tubular member 308 would typically enclose the guide tube. Segments of the guide tube (e.g., 310b, 310c, 310d, and 310e) also extend beyond the end of the fluid supply lumen.

A first inflatable structure 302 is mounted near the distal end of the catheter. This first inflatable structure 304 may have any suitable shape and size so as to fit into a main body vessel and expand a stent against such main vessel's interior wall. In the illustrated embodiment, the first inflatable structure 302 is in the form of a substantially, non-compliant balloon having a cylindrical shape. A second inflatable structure 304 is substantially compliant and mounted on a portion of the first inflatable structure 302. This second inflatable structure 304 may have any suitable shape and size so as to fit into a body vessel branch. As shown, the second inflatable structure 304 forms a toroid shape around a middle region of the first inflatable structure 302. Alternatively, the second inflatable structure 304 may have a partial toroid shape (e.g., half toroid that encompasses half of the first inflatable structure's circumference).

A toroid shaped inflatable structure may be formed by compliant material that covers the entire toroid or partial toroid shape to form an opening through such toroid. In this embodiment, the first inflatable structure is inserted through such opening and adheres to the interior opening region of the second inflatable structure. In an alternative example, a compliant material only partially covers a toroid or partial toroid shape and adheres to the first inflatable structure along two circumferences, e.g., 306a and 306b (or partial circumferences). In this latter case, the inside of the toroid is completely open with respect to the underlying first balloon portion that resides between these two circumferences. In either case, the second inflatable structure 304 is adhered to the first inflatable structure using any suitable adhesive.

In the illustrated example, the fluid supply lumen(s) open into the first non-compliant balloon 302 to facilitate inflation of both the non-compliant and compliant balloon. Both inflatable balloon are in fluid communication with at least one fluid supply lumen. In this embodiment, the balloons may be coupled to the same fluid supply lumen so that they are inflated simultaneously (as illustrated in FIGS. 3A through 3D).

When the same one or more inflation lumen(s) are used to inflate both the first and second inflatable structures, the first and second balloons are arranged in fluid communication with each other. In one implementation, the same inflation lumen is positioned inside the first balloon and the inside of the first balloon is in fluid communication with the inside of the second balloon. To achieve fluid communication between the first and second balloons, the first balloon may have one or more opening(s) that are aligned with one or more corresponding opening(s) in the second balloon. FIG. 3C illustrates the plurality of openings in the non-compliant balloon 302 for fluid communication between the non-compliant and compliant balloons in accordance with one implementation of the present invention. In this implementation, the non-compliant balloon has a plurality of openings 318a through 318g evenly distributed along a same circumference. The compliant balloon can be adhered to the non-compliant balloon so that the compliant balloon's interior is completely open to the openings 318 of the non-compliant balloon. Alternatively, the compliant balloon can form an enclosed toroid that has interior openings that are aligned with the openings 318 of the non-compliant balloon. In this embodiment, the interior hole of the compliant, toroid balloon is adhered to the non-compliant balloon portion that is between the circumferences 306a and 306b so as to form a seal around each of the openings of the first and second balloons.

FIGS. 3D through 3H illustrate different lumen configurations for the catheter of FIGS. 3A through 3C in accordance with various embodiments of the present invention. Each configuration is a cross section of a tubular member, such as member 308 of FIGS. 3A through 3C. In all of the configuration examples, one or more lumen are formed within a flexible tubular member 350. The guide lumen 354 is shown as a tube having a circular cross section although it could be any suitable shape. In each of these configuration examples, a single fluid lumen 352 is utilized although any number of fluid lumen may be utilized. The cross section shape may be any suitable shape, such as shape 352a, 352b, 352c, 352d, or 352e.

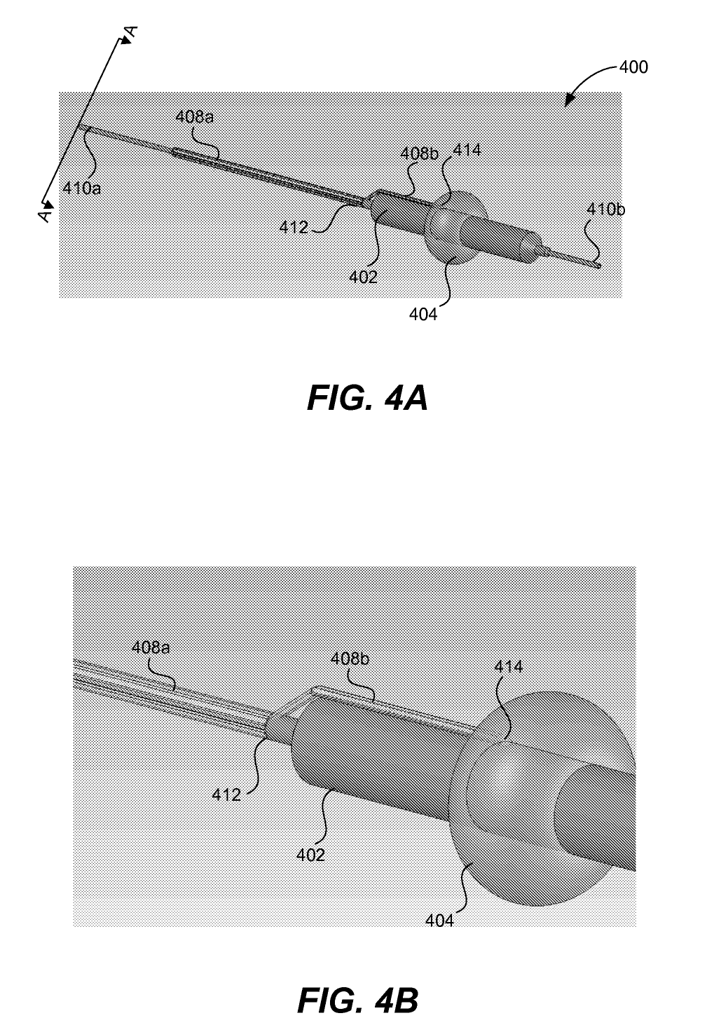

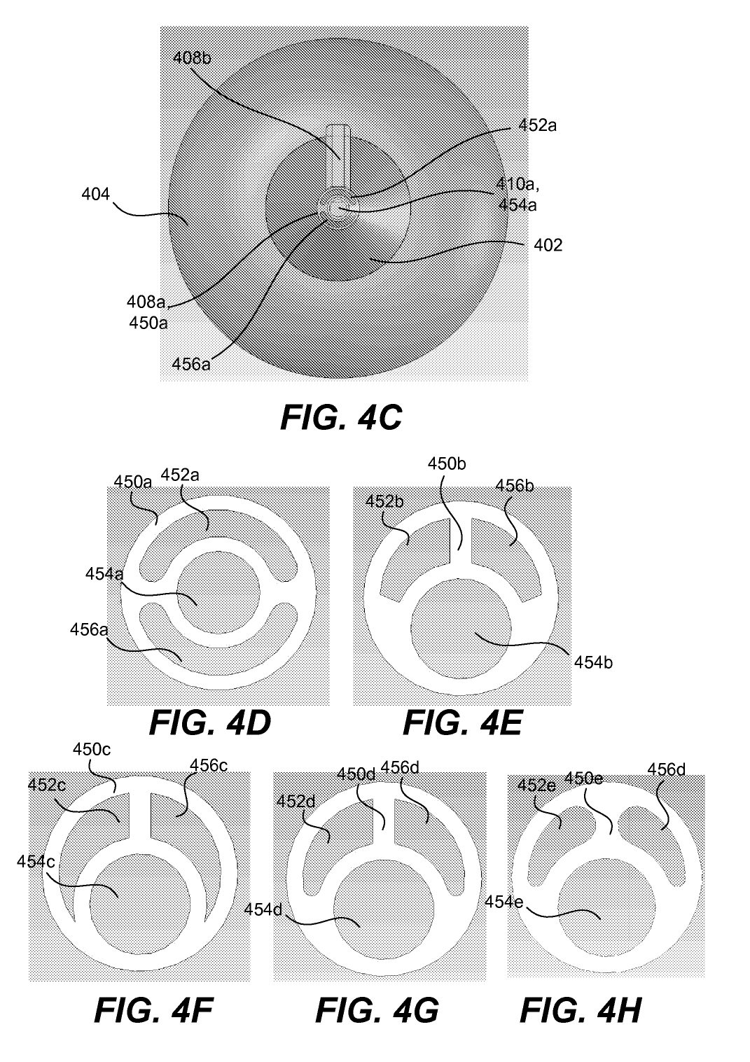

In an alternative fluid configuration, different inflation lumens are used to inflate the non-compliant balloon and the compliant balloon. FIG. 4A is a diagrammatic representation of a catheter 400 having a dual balloon mechanism with dual inflation lumen in accordance with one embodiment of the present invention. FIG. 4B is a close up of the dual balloon mechanism of FIG. 4A. As in the single inflation lumen example of FIGS. 3A through 3H, the catheter 400 includes an elongate tubular member 408a that contains at least one guide tube or lumen 410. In contrast, this elongate tubular member also contains at least two inflation lumen. A first inflation lumen inside tubular member 408a extends at least to an end 412 of the non-compliant balloon 402, and a second inflation lumen is contained within a second tubular portion 408b that extends off of the first tubular member 408a. The second inflation lumen of the second tubular member 408b extends to a position 414 inside a second compliant balloon 404. FIG. 4C is a cross section of the catheter of FIG. 4A along line A-A that illustrates the lumen configuration relative to the two balloons in accordance with one embodiment of the present invention.

FIGS. 4D through 4H illustrate different lumen configurations for the catheter of FIGS. 4A and 4B in accordance with various embodiments of the present invention. Each configuration is a different cross section example of a tubular member, such as 308 of FIGS. 4A and 4B. In all of the configuration examples, one or more lumen are formed within a flexible tubular member 450. The guide lumen 454 is shown as a tube having a circular cross section although it could be any suitable shape. In each of these configuration examples, two fluid lumen 452 and 456 are utilized although any number of fluid lumen may be utilized. The cross section shape may be any suitable shape, such as lumen pairs 452a and 456a, 452b and 456b, 452c and 456c, 452d and 456d, and 452e and 456e. Lumen pair 452a and 456a and guide lumen 454a of tubular member 450a are also illustrated in FIG. 4C.

In the illustrated embodiments, the guide wire lumen extends beyond the fluid supply lumens. In this embodiment, the non-compliant balloon may be attached to the flexible tubular member/guide wire lumen at any appropriate position, as for example along the length of the balloon, at their distal and proximal ends or the like. The balloons may be attached to each other and/or the flexible tubular member/guide wire lumen by any suitable mechanism, as for example, by an adhesive, welding, ultrasonic welding, rotation welding, RF energy, laser welding, white light welding, or mechanical bonding.

It should be appreciated that in many medical applications (e.g., most stent delivery and angioplasty applications) it is generally desirable to provide an inflatable structure that has relatively uniform expansion in all directions. Various sheaths and other arrangements can be used to encourage the individual balloons to adopt a profile that is closer to the illustrated shape than an ordinary balloon might naturally adopt.

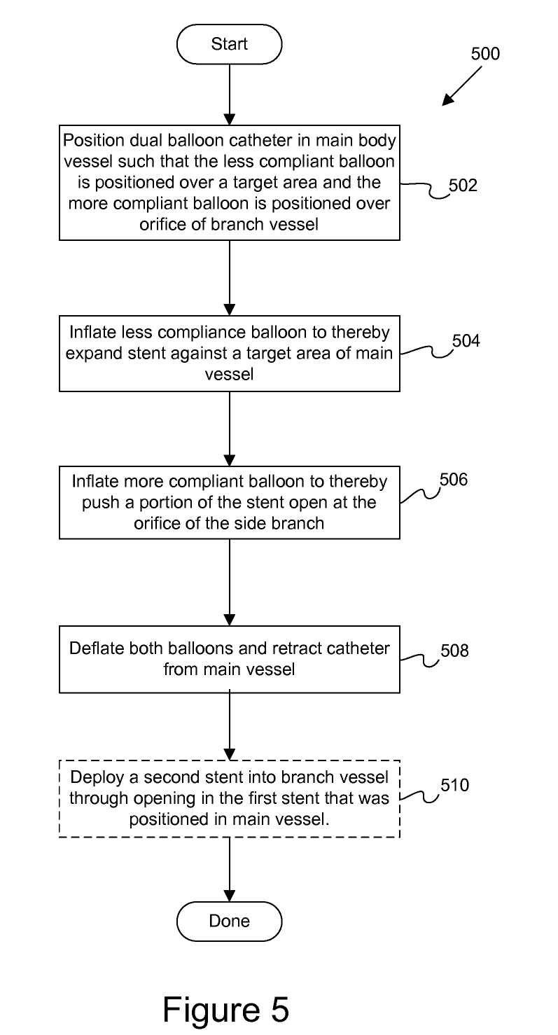

FIG. 5 is a flowchart illustrating a procedure for deploying a stent using a dual balloon system in accordance with one embodiment of the present invention. Initially, a dual balloon (or any other suitable non-compliant and compliant inflation structure arrangement) having a stent mounted thereon is positioned in a main body vessel such that the less compliant balloon is positioned adjacent to target area in the main vessel and the more compliant balloon is positioned over the orifice of a branch vessel in operation 502. This positioning operation may be accomplished in any suitable manner. For example, each balloon may include one or more markers that are detectable with an detection system. In a specific example, two markers are used for each balloon to mark the end points of each balloon. Alternatively, a single marker may be positioned on one or both of the balloons to denote the central position of one or both of the balloons. As shown in FIG. 3B, a pair of markers 324a and 324b are used to denote the ends of the non-compliant balloon 302, while a single marker 322 is used to denote to middle (dashed line) of the compliant balloon 304. Of course, a single marker may be used for the non-compliant balloon and a pair of markers may be used for the compliant balloon. Additionally, if both of the balloons have a common center, such center may be marked with a single marker and positioned over a central target area, for example.

The markers may be formed from a metallic material, such as gold or palladium and imaged using an X-ray imaging system. The markers are used to position the balloons relative to the target area and the opening in the branch vessel. If the compliant balloon forms a partial toroid shape (or asymmetrical shape), the markers may also be used to align the partial toroid portion with the opening.

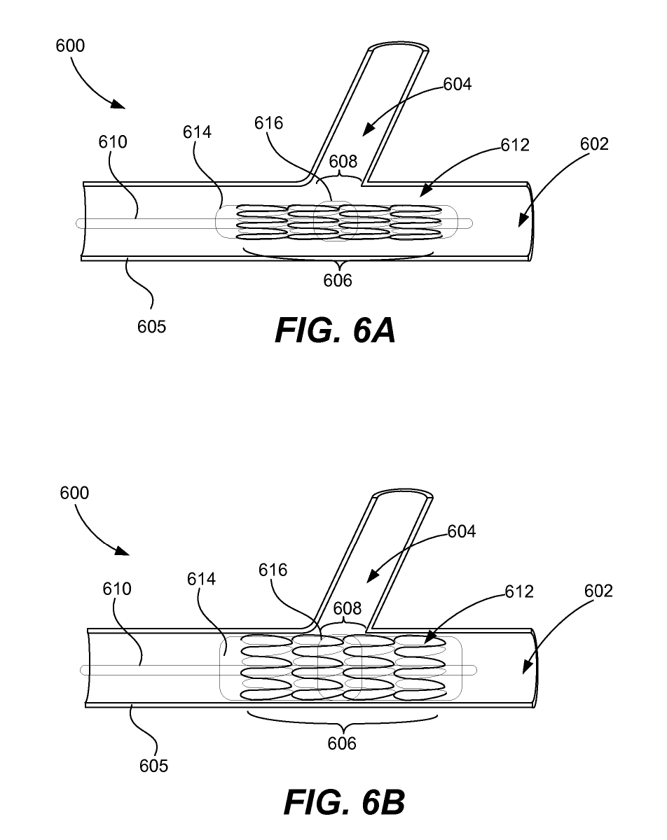

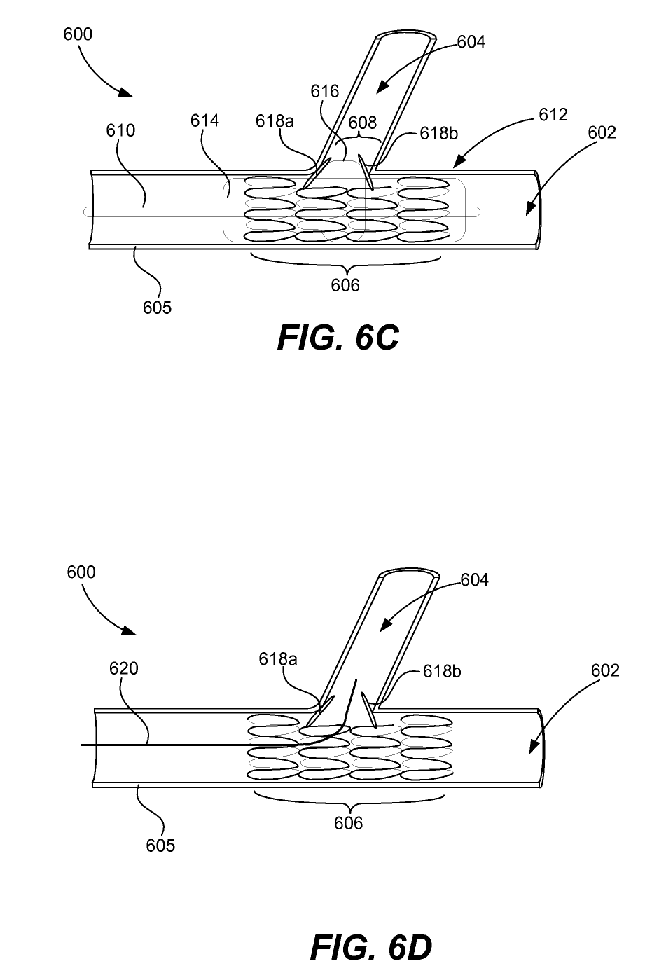

FIG. 6A is a diagrammatic representation illustrating positioning of a catheter having a dual balloon and stent within a bifurcated vessel in accordance with one embodiment of the present invention. As shown, a catheter 612 is positioned along main vessel 602. The catheter includes an elongated, flexible member 610 that supports a non-compliant balloon 614 and compliant balloon 616. A stent 606 is crimped around both balloons, and the stent is positioned along a target area of the main vessel. The compliant balloon 616 is positioned over the opening 608 of the branch vessel 604.

After the balloons are positioned, the less compliant balloon may be inflated to thereby expand the stent at the target area in operation 504. FIG. 6B illustrates inflation and expansion of the non-compliant balloon 614 and overlying stent 606 of FIG. 6A. As shown, the stent 606 pushes against and supports the target area of the main vessel's wall 605. The more compliant balloon is also inflated (simultaneously or separately with inflation of the less compliant balloon) to thereby push a portion of the stent open at the orifice of the branch vessel in operation 506. FIG. 6C illustrates inflation and expansion of the compliant balloon 616. Stent portions 618a and 618b open into opening 608 of branch 604. It should be noted that the symmetrical compliant balloon 616 expands asymmetrically since a first side is pressed against the main vessel wall 605 while the second side expands into the branch vessel 604.

After the stent is opened across the branch vessel, the catheter may then be retracted from the main vessel in operation 508. FIG. 6D illustrates a main vessel into which a stent has been deployed in a bifurcated vessel so that the stent opens across the branch vessel in accordance with one embodiment of the present invention. As shown, the stent portions 618 remain open across branch vessel 604, while the remaining stent portions 606 remain against the main vessel side wall 605. Another guide wire 620 (or any other medical device) may also be inserted through such opening in operation 510.

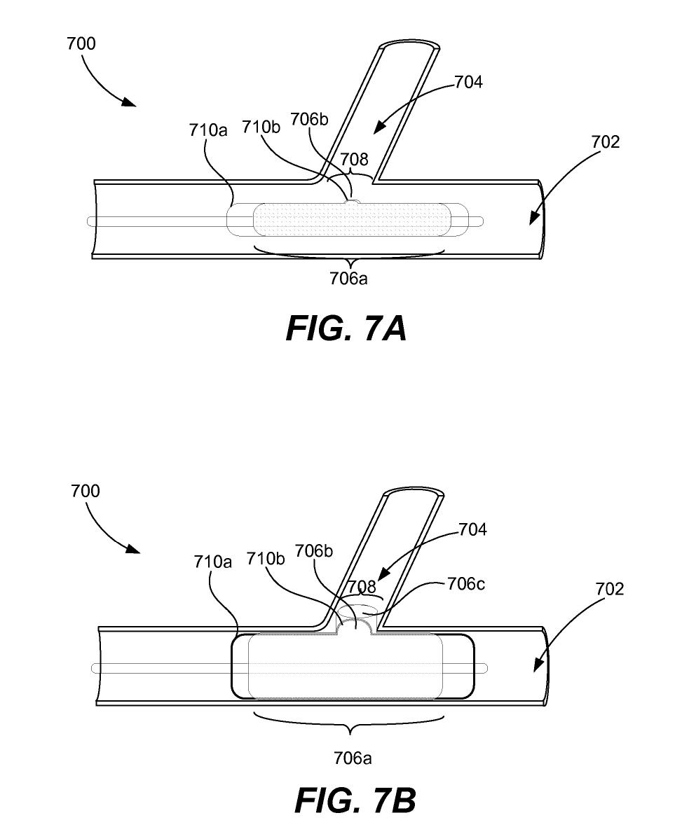

FIGS. 7A and 7B illustrate an alternative stent and balloon arrangement 700 for deploying in a bifurcated vessel. As shown in FIG. 7A, a balloon 710 having a first portion 710a for inflating in a main vessel 702 and a second portion 710b for inflating into a side vessel portion 704 may be utilized to expand a stent 706 having a first portion 706a for expanding in the main vessel 702 and a second extension portion 706b for extending and opening into the side vessel 704 through opening 708. The second extension portion 706b of the stent is a deformable feature that is extendable into the side branch as shown in FIG. 7B. The second balloon portion 710b is a collapsed nub or nipple in the balloon 710 when the balloon is in a collapsed state as shown in FIG. 7A, and this balloon nub 710b extends upon expansion and pushes the second extension portion 706b of the stent into the side vessel 704 as shown in FIG. 7B. A guide wire or any other medical device may also be inserted through an opening 706c of the extension stent portion 706b so as to inserted into the side vessel 704. Alternatively, any of the dual balloons described herein may be utilized with the stent of FIGS. 7A and 7B, or any of the stents described herein may be utilized with the balloon of FIGS. 7A and 7B.

Embodiments of the present invention have several associated advantages. Using a compliant balloon to form a stent opening across a branch blood vessel minimizes disturbances in the blood flow through such branch vessel due a decrease in stent jailing across the branch opening, as well reducing plaque deposits becoming entrapped in stent portions that block the branch opening. Additionally, insertion of a guide into the branch opening after stent deployment is facilitated by such stent opening.

It should be appreciated that the described catheter arrangement can be useful in a wide variety of interventional procedures. For example, it may be useful in applying stents to one or both branches of a vessel bifurcation. Alternatively, the described arrangements may be useful in facilitating appropriate diagnostic or treatment procedures in a branch of a bifurcation, either together with or separate from a procedure that might be performed in the main branch. The procedures may include such procedures as angioplasty procedures, atherectomy procedures, stent delivery procedures, localized drug delivery procedure, visualization procedures, tissue or fluid (e.g., blood) sample acquiring procedures, etc.

Although the foregoing invention has been described in some detail for purposes of clarity of understanding, it will be apparent that certain changes and modifications may be practiced within the scope of the appended claims. Although the stent opening has been described primarily in the context of permitting a guide wire to pass there through into a side branch of a bifurcation, it should be appreciated that a variety of small sized tools may be passed through the stent opening in addition to or in place of a guide wire. The described structure may be incorporated into a simple angioplasty or stent delivery catheter or into different and/or more complicated medical devices. Therefore, the present embodiments are to be considered as illustrative and not restrictive and the invention is not to be limited to the details given herein, but may be modified within the scope and equivalents of the appended claims.

What is claimed is:

1. A catheter comprising:
an elongate flexible tubular member having a plurality of lumens including a guide lumen and at least one inflation lumen, the flexible tubular member being sized for insertion in a body vessel;
a first inflatable structure carried by a distal portion of the flexible tubular member, the first inflatable structure being in fluid communication with at least one of the inflation lumen, wherein the first inflatable structure is formed from a substantially non-compliant material; and
a second inflatable structure carried by a distal portion of the flexible tubular member, the second inflatable structure being in fluid communication with at least one of the inflation lumen, wherein the second inflatable structure is formed from a substantially, compliant material.

2. A catheter as recited in claim 1, wherein the second inflatable structure covers a portion of the first inflatable structure.

3. A catheter as recited in claim 2, wherein the second inflatable structure forms a toroid shape that encompasses a portion of the first inflatable structure.

4. A catheter as recited in claim 2, wherein the second inflatable structure forms a partial toroid shape that partially encompasses the first inflatable structure.

5. A catheter as recited in claim 1, wherein the first and second inflation structures are in fluid communication with the same inflation lumen.

6. A catheter as recited in claim 5, wherein the same inflation lumen is positioned inside the first inflation structure and the inside of the first inflation structure is in fluid communication with the inside of the second inflation structure.

7. A catheter as recited in claim 6, wherein first inflation structure has one or more opening(s) that are in fluid communication with an interior of the second inflation structure.

8. A catheter as recited in claim 7, wherein each opening is aligned with a corresponding opening in the second inflation structure and wherein a portion of the first inflation structure is adhered to a portion of the second inflation structure so as to form a seal around each of the openings of the first and second inflation structures.

9. A catheter as recited in claim 8, wherein the openings of the first inflation structures are radially distributed around the hole of the second inflation structure.

10. A catheter as recited in claim 1, wherein the first inflation structure is in fluid communication with a first inflation lumen and the second inflation structure is in fluid communication with a second inflation lumen that differs from the first inflation lumen.

11. A catheter as recited in claim 10, wherein the first inflatable structure has a cylindrical shape and the second inflatable structure is formed on an external, curved surface portion of the first inflatable structure, and wherein the first inflation lumen extends to inside an end of the first inflation structure and the second inflation lumen branches from the tubular member and extends to the second inflatable structure.

12. A catheter as recited in claim 1, further including a plurality of markers for detecting a position of the first inflatable structure and a position of the second inflatable structure.

13. A catheter as recited in claim 1, wherein the first inflatable structure is formed from a material selected from a group consisting of Nylon, Pebax, PEEK (Polyetheretherketone), PET (Polyethylene Terephthalate), and PTFE (Polytetrafluoroethylene), and the second inflatable structure is formed from a material selected from a group consisting of Pebax, C-Flex, Latex, Polyethylene, PE600, Urethane, and Silicone.

14. A catheter as recited in claim 1, wherein the guide lumen is sized for insertion of a guide wire or exchanger.

15. A catheter as recited in claim 1, wherein the first and second inflatable structures are integrated together so that the second inflatable structure is a nub on the first inflatable structure, the catheter further comprising a stent having a first portion for positioning over the first inflatable portion and a second extendable portion for positioning over the second inflatable structure so that the second extendable portion pushes out and forms an opening when the second inflatable structure is inflated.

16. A method for inserting a stent into a body vessel using a catheter, wherein the catheter comprises a first inflatable structure carried by a distal portion of a flexible tubular member and a second inflatable structure carried by a distal portion of the flexible tubular member, wherein the first inflatable structure is formed from a substantially, non-compliant material and the second inflatable structure is formed from a substantially, compliant material, the method comprising:
positioning a stent over a portion of the first and second inflatable structures and positioning the catheter in a main vessel such that the first inflatable structure is positioned along a target area of the main vessel and the second inflatable structure is positioned across an orifice of a branch vessel that branches from the main vessel;
inflating the first inflatable structure to thereby expand the stent against the target area of the main vessel;
inflating the second inflatable structure to thereby push a portion of the stent to form an opening in the stent portion at the orifice of the branch vessel; and
deflating the first and second inflatable structures and removing the catheter from the main vessel.

17. A method as recited in claim 16, further comprising deploying a second stent into the branch vessel through the opening of the first stent.

18. A method as recited in claim 16 wherein the operation of inflating the first inflatable structure is performed substantially, simultaneously with the operation of inflating the second inflatable structure.

19. A method as recited in claim 16, wherein the operation of inflating the first inflatable structure is performed prior to the operation of inflating the second inflatable structure.
